(19) United States
(12) Patent Application Publication
(10) Pub. No.: US 2019/0289179 A1
(43) Pub. Date: Sep. 19, 2019

(54) ENDOSCOPE IMAGE PROCESSING DEVICE AND ENDOSCOPE IMAGE PROCESSING METHOD

(71) Applicant: OLYMPUS CORPORATION, Tokyo (JP)

(72) Inventor: Motohiro MITAMURA, Tokyo (JP)

(73) Assignee: OLYMPUS CORPORATION, Tokyo (JP)

(21) Appl. No.: 16/427,447

(22) Filed: May 31, 2019

Related U.S. Application Data
(63) Continuation of application No. PCT/JP2017/001050, filed on Jan. 13, 2017.

(51) Int. Cl. H04N 5/225; A61B 1/00; A61B 1/06

(52) U.S. Cl. CPC H04N 5/2256; A61B 1/0638; A61B 1/0005; A61B 1/00009

(57) ABSTRACT

The present invention provides an endoscope image processing device that processes a normal-light image of a subject illuminated with broadband visible light and a special-light image of the subject illuminated with narrow-band special light, the endoscope image processing device including: a non-structure reducing unit that reduces non-structure information having no correlation with the structure of the subject, in the special-light image; a superimposed-image generating unit that generates a superimposed image by superimposing the special-light image in which the non-structure information has been reduced, on the normal-light image; and an output unit that outputs the superimposed image to an external device.

### CROSS-REFERENCE TO RELATED APPLICATIONS

This is a continuation of International Application PCT/JP2017/001050, with an international filing date of Jan. 13, 2017, which is hereby incorporated by reference herein in its entirety.

### TECHNICAL FIELD

The present invention relates to an endoscope image processing device and an endoscope image processing method.

### BACKGROUND ART

In the related art, there is a known endoscope device that obtains a normal-light image, such as a white-light image, and a special-light image, such as a fluorescence image, that superimposes the special-light image on the normal-light image, and that displays the superimposed image (for example, see Publication of Japanese Patent No. 4799109).

### SUMMARY OF INVENTION

According to one aspect, the present invention provides an endoscope image processing device that processes a normal-light image of a subject illuminated with broadband visible light and a special-light image of the subject illuminated with narrow-band special light, the endoscope image processing device including: a non-structure reducing unit that reduces non-structure information having no correlation with the structure of the subject, in the special-light image; a superimposed-image generating unit that generates a superimposed image by superimposing the special-light image in which the non-structure information has been reduced by the non-structure reducing unit, on the normal-light image; and an output unit that outputs the superimposed image generated by the superimposed-image generating unit, to an external device.

In the above-described aspect, the non-structure reducing unit may reduce the brightness of the special-light image.

In the above-described aspect, the non-structure reducing unit may thin out some pixels of the special-light image.

In the above-described aspect, the non-structure reducing unit may make the pixels to be thinned out different between a plurality of time-series special-light images.

In the above-described aspect, the non-structure reducing unit may selectively reduce the non-structure information, without reducing structure information of the subject.

The above-described aspect may further include a structure enhancement unit that enhances structure information of the subject included in the normal-light image.

According to another aspect, the present invention provides an endoscope image processing method for processing a normal-light image of a subject illuminated with broadband visible light and a special-light image of the subject illuminated with narrow-band special light, the endoscope image processing method including the steps of: reducing non-structure information having no correlation with the structure of the subject, in the special-light image; generating a superimposed image by superimposing the special-light image in which the non-structure information has been reduced, on the normal-light image; and outputting the generated superimposed image to an external device.

### BRIEF DESCRIPTION OF DRAWINGS

FIG. 1 is a block diagram showing the functions of an endoscope image processing device according to one embodiment of the present invention.

FIG. 2A is a view showing an example normal-light image generated by a normal-light image generating unit of the endoscope image processing device shown in FIG. 1.

FIG. 2B is a view showing an example fluorescence image generated by a fluorescence image generating unit of the endoscope image processing device shown in FIG. 1.

FIG. 3 is a view for explaining pixel thinning-out processing performed by a non-structure reducing unit of the endoscope image processing device shown in FIG. 1.

FIG. 4 is a flowchart showing an endoscope image processing method performed by the endoscope image processing device shown in FIG. 1.

FIG. 5 is a view showing example thinning-out patterns used in the pixel thinning-out processing in a first modification of the endoscope image processing device shown in FIG. 1.

FIG. 6 is a block diagram showing the functions in a second modification of the endoscope image processing device shown in FIG. 1.

FIG. 7 is a block diagram showing the functions in a third modification of the endoscope image processing device shown in FIG. 1.

FIG. 8 is a block diagram showing the functions in a fourth modification of the endoscope image processing device shown in FIG. 1.

### DESCRIPTION OF EMBODIMENTS

An endoscope image processing device 1 according to one embodiment of the present invention will be described below with reference to the drawings.

The endoscope image processing device (hereinafter, simply referred to as “image processing device”) 1 of this embodiment is connected to an endoscope device and a display device (external devices), sequentially receives, from the endoscope device, image signals acquired by the endoscope device, generates a superimposed image, to be described later, by processing the received image signals, outputs the generated superimposed image to the display device, and causes the display device to display the superimposed image.

The endoscope device radiates normal light onto a living tissue (subject) and captures reflected light of the normal light from the living tissue by means of an image-acquisition element, thereby acquiring a normal-light image signal. The endoscope device radiates excitation light onto the living tissue and captures fluorescence produced by a fluorescent substance in the living tissue by means of the image-acquisition element, thereby acquiring a fluorescence image signal. The normal light is broadband visible light, such as white light, and the excitation light is narrow-band light. The fluorescent substance is, for example, a drug that accumulates in a particular area, such as a lesion, in the living tissue.

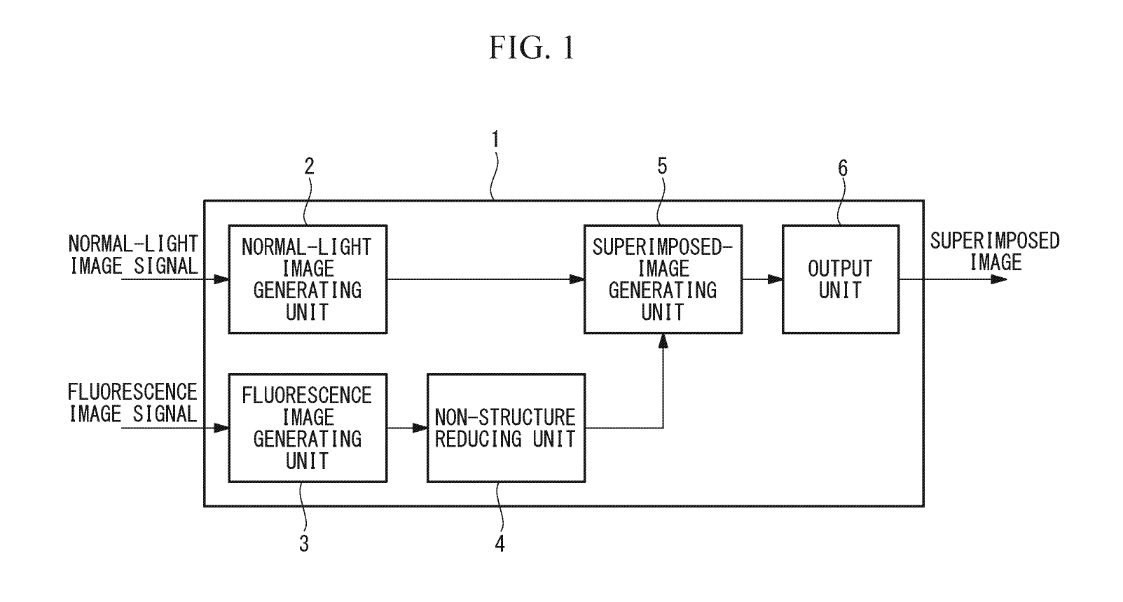

As shown in FIG. 1, the image processing device 1 is provided with a normal-light image generating unit 2, a fluorescence image generating unit 3, a non-structure reducing unit 4, a superimposed-image generating unit 5, and an output unit 6.

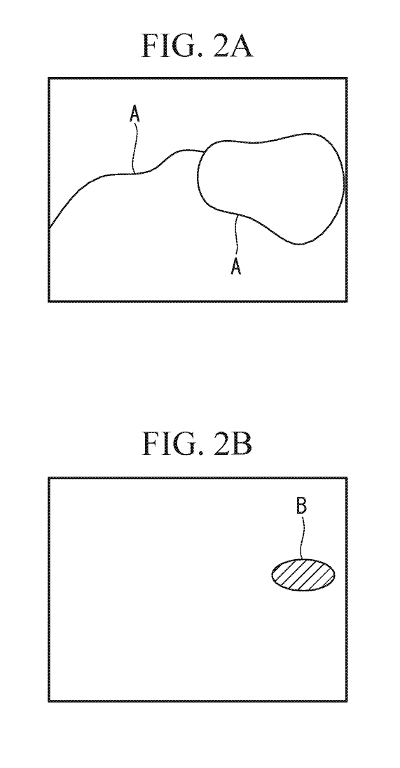

The normal-light image generating unit 2 receives a normal-light image signal from the endoscope device, generates a normal-light image from the normal-light image signal, and sends the normal-light image to the superimposed-image generating unit 5. As shown in FIG. 2A, the normal-light image is an image expressing the structure of living tissue, such as the surface form of living tissue. Therefore, the normal-light image includes a lot of structure information that has a spatial correlation with the structure of the living tissue, such as outlines A of the living tissue.

The fluorescence image generating unit 3 receives a fluorescence image signal from the endoscope device, generates a fluorescence image (special-light image) from the fluorescence image signal, and sends the fluorescence image to the non-structure reducing unit 4. The fluorescence image is an image acquired by capturing fluorescence from a particular area, such as a lesion. Therefore, as shown in FIG. 2B, the fluorescence image is an image including a lot of non-structure information that has no or low spatial correlation with the structure of the living tissue, as in a fluorescence area B.

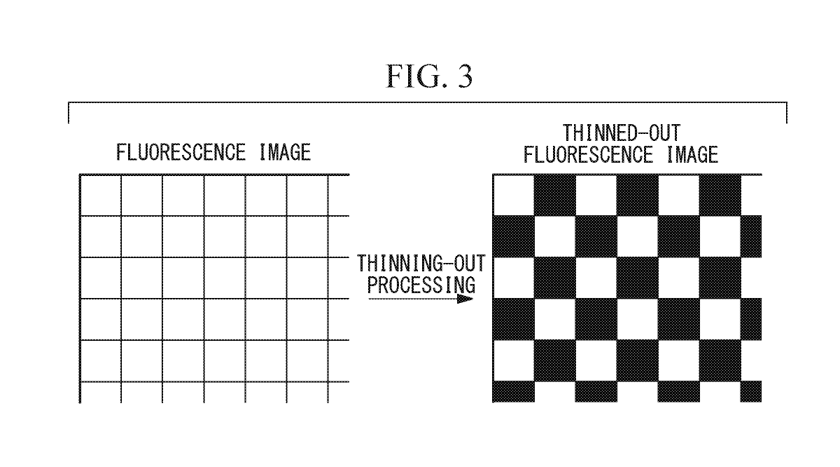

The non-structure reducing unit 4 uniformly thins out pixels over the entirety of the fluorescence image, generates a thinned-out fluorescence image (see the right view in FIG. 3) in which some pixels are missing, and sends the thinned-out fluorescence image to the superimposed-image generating unit 5. For example, as shown in FIG. 3, the non-structure reducing unit 4 thins out pixels, in an alternate manner, in the row direction and in the column direction. In FIG. 3, white squares indicate pixels, and black squares indicate thinned-out pixels.

The normal-light image is, for example, a color image having an RGB format and has red (R), green (G), and blue (B) channels. The superimposed-image generating unit 5 adds the signal of the thinned-out fluorescence image to a G-channel signal of the normal-light image, thereby generating a superimposed image in which the thinned-out fluorescence image is superimposed on the normal-light image, and sends the superimposed image to the output unit 6. The thinned-out fluorescence image may also be added to an R- or B-channel, instead of the G-channel.

The output unit 6 outputs the superimposed image to the display device at a fixed frame rate.

The image processing device 1 is realized by, for example, a computer that is provided with a central processing unit (CPU) and a storage device that stores an image processing program for causing the CPU to execute the processing of the above-described respective units 2, 3, 4, and 5.

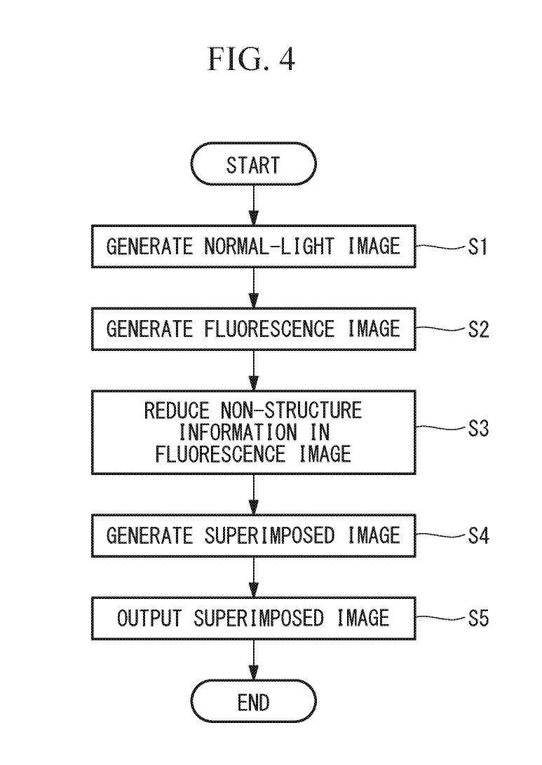

Next, the operation of the image processing device 1 will be described with reference to FIG. 4.

A normal-light image signal and a fluorescence image signal that are acquired by the endoscope device are sequentially input to the image processing device 1. In the image processing device 1, a normal-light image is generated from the normal-light image signal in the normal-light image generating unit 2 (Step S1), and a fluorescence image is generated from the fluorescence image signal in the fluorescence image generating unit 3 (Step S2).

Next, in the non-structure reducing unit 4, a thinned-out fluorescence image is generated by thinning out some pixels in the fluorescence image (Step S3). Next, in the superimposed-image generating unit 5, a superimposed image is generated by adding the thinned-out fluorescence image to the G-channel of the normal-light image (Step S4). The generated superimposed image is sent to the display device via the output unit 6 and is displayed on the display device (Step S5).

In this way, according to this embodiment, the fluorescence image in which the non-structure information, such as the fluorescence area B, has been reduced by thinning out some pixels is used in generating a superimposed image, thus generating a superimposed image that has, in a mixed manner, pixels that have the normal-light image signal as is and pixels obtained by adding the fluorescence image signal to the normal-light image signal. Accordingly, there is an advantage in that it is possible reduce deterioration of the structure information in the normal-light image due to the non-structure information in the fluorescence image and to generate a superimposed image in which the structure information of living tissue in the normal-light image is clearly maintained.

Next, modifications of the image processing device 1 of this embodiment will be described. First to fourth modifications, described below, may be appropriately combined and realized.

### First Modification

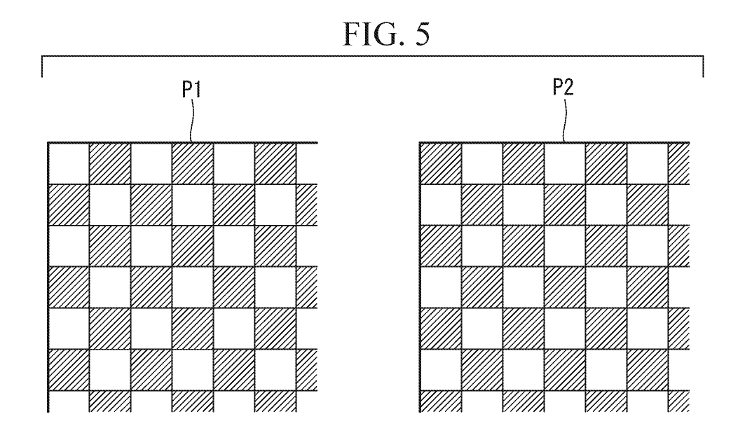

In an image processing device according to a first modification, the non-structure reducing unit 4 has a plurality of thinning-out patterns P1 and P2 in which the positions of thinning-out target pixels to be thinned out from a fluorescence image are specified, as shown in FIG. 5. In FIG. 5, hatched pixels indicate thinning-out target pixels. Although two types of the thinning-out patterns P1 and P2 are shown in FIG. 5, it is also possible to prepare three or more types of thinning-out patterns.

The plurality of thinning-out patterns P1 and P2 are designed such that the positions of thinning-out target pixels are made different from each other. The non-structure reducing unit 4 applies, in turn, the plurality of thinning-out patterns P1 and P2 to a plurality of time-series fluorescence images received from the fluorescence image generating unit 3, to generate thinned-out fluorescence images. Accordingly, thinned-out fluorescence images having the plurality of patterns, in which the pixel thinning-out positions are different from each other, are generated in turn. When superimposed images generated by using the thinned-out fluorescence images having such a plurality of patterns are displayed in turn on the display device, display and non-display of information at the respective positions in the fluorescence images are alternately repeated.

In a case in which the positions of thinning-out target pixels are fixed, superimposed images in which information at the same positions in fluorescence images is missing are kept so as to be provided to an observer. According to this modification, there is an advantage in that the positions of thinning-out target pixels are changed with time, thereby making it possible to provide the observer with information at all positions in fluorescence images, while reducing non-structure information in the fluorescence images.

### Second Modification

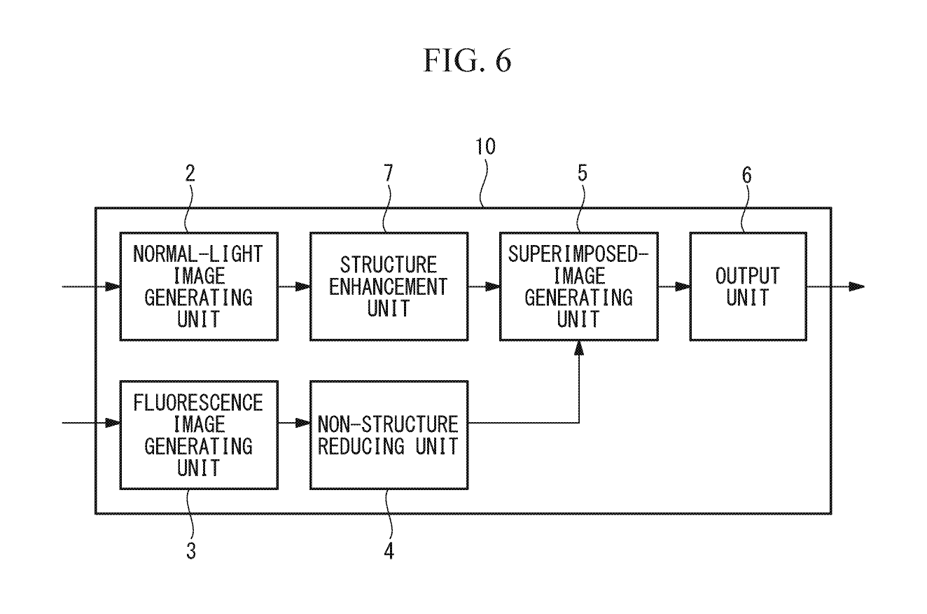

As shown in FIG. 6, an image processing device 10 according to a second modification is further provided with a structure enhancement unit 7 that performs structure enhancement processing on a normal-light image. The structure enhancement processing is processing for increasing structure information included in a normal-light image, and is, for example, edge enhancement processing or processing for increasing brightness. The superimposed-image generating unit 5 uses a normal-light image in which the structure has been enhanced by the structure enhancement unit 7, to generate a superimposed image. According to this modification, it is possible to obtain a superimposed image in which the structure of living tissue is clearer.

### Third Modification

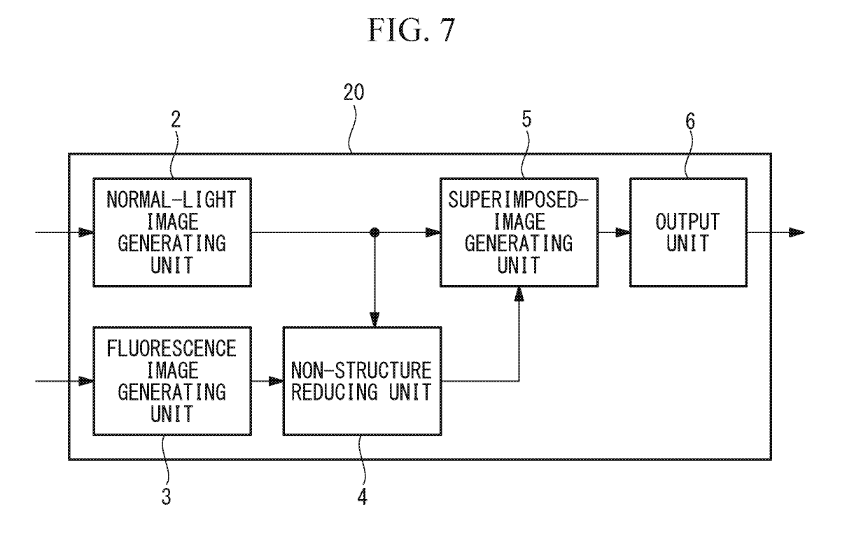

In an image processing device 20 according to a third modification, the non-structure reducing unit 4 reduces non-structure information by reducing the brightness of a fluorescence image such that the ratio of the brightness of the fluorescence image to the brightness of a normal-light image becomes a predetermined threshold or less. Specifically, as shown in FIG. 7, the non-structure reducing unit 4 receives a normal-light image from the normal-light image generating unit 2, calculates the brightness of the normal-light image, receives a fluorescence image from the fluorescence image generating unit 3, and calculates the brightness of the fluorescence image. The brightness of an image is, for example, the average value of signals at all pixels. The predetermined threshold is set such that, in a superimposed image, the structure information of the living tissue in the normal-light image is not embedded in the non-structure information in the fluorescence image. The superimposed-image generating unit 5 uses a fluorescence image whose brightness has been reduced by the non-structure reducing unit 4, to generate a superimposed image.

Although a fluorescence image can include structure information in addition to non-structure information, the structure information is less compared with the non-structure information. Therefore, when the brightness of the fluorescence image is reduced, the degree of reduction of the non-structure information becomes larger compared with the degree of reduction of the structure information, thus obtaining an effect of reducing the non-structure information relative to the structure information.

In this way, it is possible to obtain an effect of reducing the non-structure information in the fluorescence image by reducing the brightness of the fluorescence image with respect to the brightness of the normal-light image.

In this modification, instead of or in addition to reducing the brightness of a fluorescence image, it is also possible to increase the brightness of a normal-light image, thereby adjusting the relative brightness between the normal-light image and the fluorescence image such that the ratio of the brightness of the fluorescence image to the brightness of the normal-light image becomes the predetermined threshold or less.

### Fourth Modification

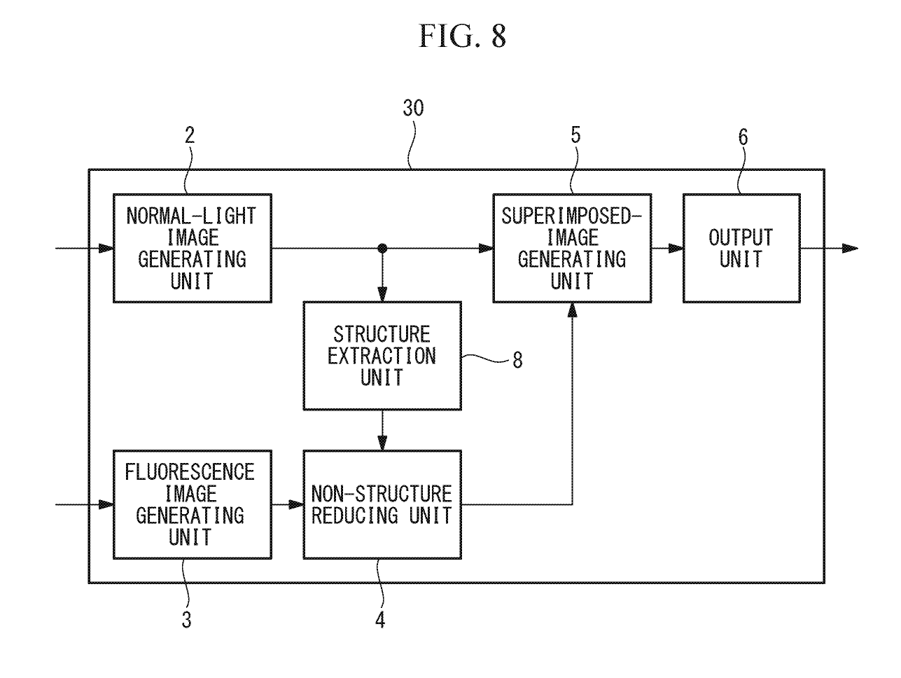

As shown in FIG. 8, an image processing device 30 of the fourth modification is further provided with a structure extraction unit 8 that extracts, from a normal-light image, a structure area having structure information such as the outline of living tissue. The structure area can be extracted, for example, through known edge extraction processing. A fluorescence image can include, in addition to the strong fluorescence area B, such as a lesion, a weak fluorescence area (i.e., structure information) along the structure of living tissue. The non-structure reducing unit 4 subtracts, from the fluorescence image, the structure area extracted by the structure extraction unit 8, thereby removing the weak fluorescence area, which extends along the structure of the living tissue, from the fluorescence image. Next, the non-structure reducing unit 4 reduces the non-structure information by thinning out some pixels from the fluorescence image, from which the structure area has been subtracted, and then, adds again the subtracted structure area to the thinned-out fluorescence image, in which the non-structure information has been reduced. Accordingly, in the fluorescence image, the non-structure information can be selectively reduced, without reducing the structure information of the subject.

In this way, the fluorescence image in which the non-structure information has been selectively reduced is used for a superimposed image, thereby making it possible to obtain an effect of enhancing, in the superimposed image, the structure information, such as the outline of living tissue.

In this modification, it is also possible to superimpose, on a normal-light image, a fluorescence image in which non-structure information has been reduced by reducing the brightness, as described in the third modification, instead of or in addition to thinning out pixels.

In the above-described embodiment and modifications, although excitation light, which excites a fluorescent substance, and a fluorescence image have been described as examples of special light and a special-light image, the types of special light and a special-light image are not limited thereto. For example, an infrared light image obtained by using infrared light or an NBI image obtained by using blue narrow-band light and green narrow-band light may also be used for superimposing on a normal-light image.

As a result, the following aspect is read from the above described embodiment of the present invention.

According to one aspect, the present invention provides an endoscope image processing device that processes a normal-light image of a subject illuminated with broadband visible light and a special-light image of the subject illuminated with narrow-band special light, the endoscope image processing device including: a non-structure reducing unit that reduces non-structure information having no correlation with the structure of the subject, in the special-light image; a superimposed-image generating unit that generates a superimposed image by superimposing the special-light image in which the non-structure information has been reduced by the non-structure reducing unit, on the normal-light image; and an output unit that outputs the superimposed image generated by the superimposed-image generating unit, to an external device.

A normal-light image of a subject illuminated with broadband visible light is an image that expresses the structure of the subject and that includes structure information of the subject. On the other hand, a special-light image thereof illuminated with narrow-band special light is an image that expresses a particular area, in the subject, reacting to the special light and that includes non-structure information having no correlation with the structure of the subject.

According to this aspect, the superimposed-image generating unit superimposes the normal-light image and the special-light image, thus generating a superimposed image in which the structure of the subject is associated with the particular area, and the generated superimposed image is output from the output unit to an external device.

In this case, because the special-light image in which the non-structure information has been reduced by the non-structure reducing unit is used for superimposing on the normal-light image, it is possible to reduce deterioration of the structure information of the subject caused when the special-light image is superimposed on the normal-light image and to generate a superimposed image in which the structure of the subject is clear.

In the above-described aspect, the non-structure reducing unit may reduce the brightness of the special-light image.

In this way, by reducing the brightness of the special-light image, it is possible to reduce the non-structure information in the superimposed image relative to the structure information, through simple processing.

In the above-described aspect, the non-structure reducing unit may thin out some pixels of the special-light image.

In this way, by thinning-out some pixels of the special-light image, it is possible to reduce the non-structure information in the special-light image through simple processing.

In the above-described aspect, the non-structure reducing unit may make the pixels to be thinned out different between a plurality of time-series special-light images.

By doing so, the positions where pixels are thinned out from special-light images are changed with time. Accordingly, when superimposed images are generated from normal-light images and special-light images, which are consecutive as in moving images, it is possible to prevent information at the same positions in the special-light images from always missing in the superimposed images and to provide an observer, who observes the superimposed images, with information at all positions in the special-light images.

In the above-described aspect, the non-structure reducing unit may selectively reduce the non-structure information, without reducing structure information of the subject.

By doing so, it is possible to obtain a special-light image in which the non-structure information is selectively reduced while maintaining the structure information of the subject. By using such a special-light image for a superimposed image, the structure information of the subject can be enhanced in the superimposed image.

The above-described aspect may further include a structure enhancement unit that enhances structure information of the subject included in the normal-light image.

By doing so, it is possible to generate a superimposed image in which the structure of the subject in the normal-light image is clearer.

According to another aspect, the present invention provides an endoscope image processing method for processing a normal-light image of a subject illuminated with broadband visible light and a special-light image of the subject illuminated with narrow-band special light, the endoscope image processing method including the steps of: reducing non-structure information having no correlation with the structure of the subject, in the special-light image; generating a superimposed image by superimposing the special-light image in which the non-structure information has been reduced, on the normal-light image; and outputting the generated superimposed image to an external device.

### REFERENCE SIGNS LIST

1, 10, 20, 30 endoscope image processing device
2 normal-light image generating unit
3 fluorescence image generating unit
4 non-structure reducing unit
5 superimposed-image generating unit
6 output unit
7 structure enhancement unit
8 structure extraction unit

1. An endoscope image processing device that comprises a controller configured to process a normal-light image of a subject illuminated with broadband visible light and a special-light image of the subject illuminated with narrow-band special light, the controller comprises a one or more processor comprising hardware, the processor being configured to:
reduce non-structure information having no correlation with the structure of the subject, in the special-light image;
generate a superimposed image by superimposing the special-light image in which the non-structure information has been reduced, on the normal-light image; and
output the superimposed image to an external device.

2. An endoscope image processing device according to claim 1, wherein the non-structure information is reduced by reducing the brightness of the special-light image.

3. An endoscope image processing device according to claim 1, wherein the non-structure information is reduced by thinning out some pixels of the special-light image.

4. An endoscope image processing device according to claim 3, wherein the non-structure information is reduced by making the pixels to be thinned out different between a plurality of time-series special-light images.

5. An endoscope image processing device according to claim 1, wherein the non-structure information is selectively reduced without reducing structure information of the subject.

6. An endoscope image processing device according to claim 1, wherein the controller further enhances structure information of the subject included in the normal-light image.

7. An endoscope image processing method for processing a normal-light image of a subject illuminated with broadband visible light and a special-light image of the subject illuminated with narrow-band special light, the endoscope image processing method comprising the steps of:
reducing non-structure information having no correlation with the structure of the subject, in the special-light image;
generating a superimposed image by superimposing the special-light image in which the non-structure information has been reduced, on the normal-light image; and
outputting the generated superimposed image to an external device.
